(19) United States
(12) Patent Application Publication
(10) Pub. No.: US 2024/0269220 A1
(43) Pub. Date: Aug. 15, 2024

(54) USES OF BLETILLA FORMOSANA EXTRACT FOR THE TREATMENT OF DISEASES ASSOCIATED WITH DYSREGULATED ACTIVATION OF NEUTROPHILS

(71) Applicants: Chang Gung University, Taoyuan City (TW); Chang Gung University of Science and Technology, Taoyuan City (TW)

(72) Inventors: Tsong-Long HWANG, New Taipei City (TW); Yu-Chia CHANG, Taichung City (TW); Wu-Dai-Yu JIANG, Taitung City, Taitung County (TW)

(21) Appl. No.: 18/566,993

(22) PCT Filed: Mar. 24, 2022
(86) PCT No.: PCT/CN2022/082715

§ 371 Date: Dec. 4, 2023

(51) Int. Cl. A61K 36/898; A61K 31/05; A61K 31/085; A61K 31/09

(52) U.S. Cl. CPC A61K 36/898; A61K 31/05; A61K 31/085; A61K 31/09

(57) ABSTRACT

Disclosed herein is a method of treating diseases and/or disorders associated with the dysregulated activation and recruitment of neutrophils. The method includes administering to a subject in need thereof an effective amount of an extract of Bletilla formosana. The Bletilla formosana extract includes, at least, compounds of 3,3′-dihydroxy-5-methoxybibenzyl (Batatacin III), 9,10-dihydro-1-[(4-hydroxyphenyl)methyl]-4-methoxy-2,7-phenanthrenediol (Orchidble), 3′,5-dimethoxy-3-hydroxybibenzyl (BF8-4-2), 3,5-dimethoxy-3′-hydroxybibenzyl (BF8-4-3) and 3-hydroxy-5-methoxybibenzyl (BF8-4-4).

### CROSS REFERENCE TO RELATED APPLICATION

“This application is a U.S. National Stage Filing under 35 U.S.C. 371 from International Patent Application Serial No. PCT/CN2022/082715, filed Mar. 24, 2022, and published on Sep. 28, 2023 under WO 2023/178592, the disclosure of which is incorporated by reference herein in its entirety.”

### BACKGROUND OF THE INVENTION

### 1. Field of the Invention

The present disclosure in general relates to the novel use of Bletilla formosana extract in treating diseases and/or disorders associated with dysregulated activation of neutrophils, such as acute respiratory distress syndrome (ARDS)

### 2. Description of Related Art

Neutrophils, the most abundant granulocytes in circulation, are responsible for eliminating pathogens through degranulation, enabling neutrophil elastase (NE) release, respiratory burst with superoxide production, and neutrophil extracellular trap (NET) formation. Thus, neutrophils are key effectors of both adaptive and innate immune systems During inflammation, adhesion and migration are both crucial steps of neutrophil recruitment, which is regulated by the conformational change of macrophage-1 antigen (Mac-1; also known as αMβ2 and CD11b-CD18) on neutrophil's surface. Dysregulated activation and recruitment of neutrophils can cause damage to host tissue through the release of excessive amounts of proteolytic enzymes, reactive oxygen species (ROS), and NETs, resulting in various morbidities, including autoimmune diseases (e.g., systemic lupus erythematosus, rheumatoid arthritis, and psoriasis), infectious diseases (e.g., sepsis), inflammatory diseases (e.g., ARDS, chronic obstructive pulmonary disease and asthma), atherosclerosis, and other major diseases (e.g., diabetes mellitus and cancers).

In traditional Chinese medicine, Bletilla tubers have been used to treat pulmonary, gastrointestinal, dermatological inflammatory and haemorrhagic diseases for thousands of years. In this application, the inventors unexpectedly discovered that Bletilla formosana extract may regulate the inflammatory condition of activated human neutrophils, thus may serve as a candidate agent for the development of a medicament for treating diseases and/or disorders associated with the dysregulated activation and recruitment of neutrophils, such as ARDS, diabetes mellitus, psoriasis, liver injury, etc.

### SUMMARY OF THE INVENTION

The present disclosure provides novel use of Bletilla formosana extract, which is found to suppress the dysregulated activated neutrophils, thus the Bletilla formosana extract may serve as a candidate agent for the development of a medicament for treating diseases and/or disorders associated with the dysregulated activation and recruitment of neutrophils, such as ARDS, acute liver injury (ALI), diabetes mellitus, or psoriasis.

Accordingly, the first aspect of the present disclosure is directed to a method of treating a subject having ARDS, ALI, diabetes mellitus, or psoriasis. The method includes administering to the subject an effective amount of the Bletilla formosana extract.

According to some embodiments of the present disclosure, the Bletilla formosana extract is prepared by,
(i) extracting Bletilla formosana bulbs, leaves, stems or a mixture thereof with water to produce a first extract and a first residue; and
(ii) extracting the first residue of step (i) with ethyl acetate to produce the Bletilla formosana extract.

According to other embodiments of the present disclosure, the Bletilla formosana extract is prepared by extracting Bletilla formosana bulbs, leaves, stems or a mixture thereof with ethyl acetate.

According to embodiments of the present disclosure, the Bletilla formosana extract includes, at least, compounds of 3,3′-dihydroxy-5-methoxybibenzyl (Batatacin III), 9,10-dihydro-1-[(4-hydroxyphenyl)methyl]-4-methoxy-2,7-phenanthrenediol (Orchidble), 3′,5-dimethoxy-3-hydroxybibenzyl (BF8-4-2), 3,5-dimethoxy-3′-hydroxybibenzyl (BF8-4-3) and 3-hydroxy-5-methoxybibenzyl (BF8-4-4).

Exemplary ARDS that may be treated by the present method includes, but is not limited to, transfusion-related lung injury, ventilator-induced lung injury, bacteria-induced lung injury, viral-induced lung injury, etc.

According to embodiments of the present disclosure, the Bletilla formosana extract is administered to the subject in the amount of 0.01 to 1,000 mg/Kg. Preferably, the Bletilla formosana extract is administered to the subject in the amount of 0.1 to 800 mg/Kg.

According to embodiments of the present disclosure, the subject suitable to be treated by the present method is a mammal; preferably, a human.

The details of one or more embodiments of this disclosure are outlined in the accompanying description below. Other features and advantages of the invention will be apparent from the detailed descriptions, and claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods, and other exemplified embodiments of various aspects of the invention. The present description will be better understood from the following detailed description read in light of the accompanying drawings, where,

FIG. 1. B. formosana extracts suppressed elastase release in stimulated human neutrophils. Neutrophils (6×10⁵ cells/mL) were incubated with 0.1% DMSO or 0.3-10 μg/mL the specified B. formosana extract for 5 min before activation with fMLF (0.1 μM) or CB (1 μg/mL) for another 10 min. The B. formosana extracts were independently prepared by extracting grounded B. formosana bulbs with (A) H (n-hexane), (B) 3H1E (n-hexane/ethyl acetate=3/1), (C) 1H1E (n-hexane/ethyl acetate=1/1), (D) EA (ethyl acetate), (E) EtOH (95% alcohol), and (F) H₂O (dd H₂O). The elastase release was measured spectrophotometrically at 405 nm. All data are displayed as mean±S.E.M. (n=6-7). ** p<0.01, *** p<0.001 as compared with the control.

FIG. 2. B. formosana extracts suppressed superoxide anions production in stimulated human neutrophils. Neutrophils (6×10⁵ cells/mL) were incubated with 0.1% DMSO or 0.3-10 μg/mL the specified B. formosana extract for 5 min before activation with fMLF (0.1 μM)/CB (1 μg/mL) for another 10 min. The B. formosana extracts were independently prepared by extracting grounded B. formosana bulbs with (A) H (n-hexane), (B) 3H1E (n-hexane/ethyl acetate=3/1), (C) 1H1E (n-hexane/ethyl acetate=1/1), (D) EA (ethyl acetate), (E) EtOH (95% alcohol), and (F) H₂O (dd H₂O). Superoxide anion production was measured using ferricytochrome c reduction method through spectrophotometry at 550 nm. All data are displayed as mean±S.E.M. (n=6-7) *p<0.05. ** p<0.01. *** p<0.001 as compared with the control.

FIG. 3. The effects of different B. formosana extracts on elastase release in stimulated human neutrophils. Human neutrophils were pretreated with 0.1% DMSO or B. formosana extracts (1, 3, and 10 μg/mL) for 5 min, and then stimulated with or without fMLF (0.1 μM)/CB (1 μg/mL) for another 10 min. The B. formosana extracts were independently prepared by extracting grounded B. formosana bulbs with (A) EA, and (B) −W+EA. The elastase release was measured spectrophotometrically at 405 nm. All data are displayed as mean±S.E.M. (n=6-8). ** p<0.01, *** p<0.001 as compared with the control.

FIG. 4. The effects of different B. formosana extracts on superoxide anions production in stimulated human neutrophils. Neutrophils (6×10⁵ cells/mL) were incubated with 0.1% DMSO or 0.3-10 μg/mL the specified B. formosana extract for 5 min before activation with fMLF (0.1 μM)/CB (1 μg/mL) for another 10 min. The B. formosana extracts include (A) EA, and (B) −W+EA extracts. Superoxide anion production was measured using ferricytochrome c reduction method through spectrophotometry at 550 nm. All data are displayed as mean±S.E.M. (n=6-8). * p<0.05, ** p<0.001, *** p<0.001 as compared with the control.

FIG. 5. B. formosana extract exhibits no cytotoxicity towards human neutrophils. Neutrophils (6×10⁵ cells/mL) were incubated with 0.1% DMSO or 0.3-10 μg/mL the specified B. formosana extract for 15 min. The B. formosana extracts were independently prepared by extracting grounded B. formosana bulbs with n-hexane (“H”), n-hexane/ethyl acetate (3/1) (“3H1E”), n-hexane/ethyl acetate (1/1) (“1H1E”), ethyl acetate (“EA”), 95% alcohol (“EtOH”), dd H₂O (“H₂O”) and the combination of dd H₂O and EA (“−W+EA”). Total LDH release was determined by treating the cells with 0.1% TX-100 for 30 min, then detecting LDH via enzyme-linked immunosorbent assay at 490 nm. All data are displayed as mean±S.E.M. (n=5).

FIG. 6. B. formosana EA extract reduced NET formation in PMA-stimulated neutrophils. Human neutrophils were pretreated with 0.1% DMSO or EA extract (1, 3, and 10 μg/mL) for 10 min and then incubated with or without 10 nM PMA for 3 hr. Then, SYTOX Green was added into the plate for 15 min. All data are displayed as mean±S.E.M. (n=3).

FIG. 7. B. formosana EA extract reduced ROS production in IMLF-stimulated neutrophils. (A) Human neutrophils were pretreated with 0.1% DMSO or EA extract (0.1, 0.3, and 1 μg/mL) for 5 min, stimulated with or without 0.1 μM fMLF for another 6 min. (B) Peak chemiluminescence and (C) area under curve (AUC) of chemiluminescence are expressed as mean±S.E.M. (n=7). * p<0.05, ** p<0.01, *** p<0.001 as compared with the control.

FIG. 8. B. formosana extract mitigated IMQ-induced psoriasis in mice. (A) Upper panels are photographs taken from one representative mouse treated with vehicle (left), IMQ (center), and IMQ+B. formosana extract (right); and (B) lower panels are the microscopic presentation of skins taken from the vehicle control, the IMQ-treated mouse, and the mouse treated with IMQ+B. formosana extract.

FIG. 9. B. formosana extract mitigated LPS-induced acute lung injury in mice. BALB/c mice (n=2-3 in each group) were treated with the vehicle (10% DMSO) or 50 mg/kg B. formosana extract through intraperitoneal injection followed by intratracheal spraying of LPS (2 mg/kg) for 5 h. Light microscopy images of the H&E-stained lung sections from mice treated with or with the B. formosana extract.

FIG. 10. B. formosana extract mitigated D-GalN/LPS-induced acute liver injury (ALI) in mice. BALB/c mice (n=2-3 in each group) were treated with the vehicle (10% DMSO) or D-GalN (400 mg/kg)+LPS (40 μg/mL) through intraperitoneal injection for 1 hr, which was followed by intraperitoneal injection of B. formosana extract (50 or 100 mg/kg) for 5 h. Blood samples were drawn and the plasma levels of (A) GOP; and (B) GPT were determined.

FIG. 11. B. formosana extract mitigated D-GalN/LPS-induced acute liver injury (ALI) in mice. BALB/c mice (n=2-3 in each group) were treated with the vehicle (10% DMSO) or D-GalN (400 mg/kg)+LPS (40 μg/mL) through intraperitoneal injection for 1 hr, which was followed by intravenous injection of B. formosana extract (50 or 100 mg/kg) for 5 h. Mice were sacrificed and their liver tissues were harvested and analyzed by H&E staining.

FIG. 12. B. formosana extract reduced blood glucose level elevation in STZ-induced diabetes mice. C57BL/6 mice (n=2-3 in each group) were treated with the vehicle (saline) or STZ (50 mg/kg) through intraperitoneal injection for 5 days Mice exhibited elevated blood glucose level were selected for subsequent treatment, in which B. formosana extract (25 mg/kg) was intraperitoneally injected for consecutive 5 days, from day 9 to day 13. The blood glucose level (A) and body weight (B) of the mice were measured from day 9 to day 13.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

The term “administered”, “administering” or “administration” are used interchangeably herein to refer a mode of delivery, including, without limitation, intravenously, intramuscularly, intraperitoneally, intraarterially, intracranially, or subcutaneously administering an agent (e.g., a compound or a composition) of the present invention.

An “effective amount” of a B. formosana extract described herein (either taken alone or in a combination of another agent) refers to an amount sufficient to elicit the desired biological response, e.g., inhibiting the activation of inflammation or alleviating a target disease described herein or a symptom associated with the disease. As will be appreciated by those of ordinary skill in this art, the effective amount of B. formosana extract described herein may vary depending on such factors as the desired biological endpoint, the pharmacokinetics of the extract, the condition being treated, the mode of administration, and the age and health of a subject. In some examples, an effective amount can be a therapeutically effective amount, which refers to an amount of a therapeutic agent, alone or in combination with other therapies, sufficient to provide a therapeutic benefit in the treatment of a condition or to delay the onset or minimize one or more symptoms associated with the condition. The therapeutically effective amount refers to an amount that improves overall therapy, reduces or avoids symptoms, signs, or causes of the condition, and/or enhances the therapeutic efficacy of another therapeutic agent. In other examples, the effective amount can be a prophylactically effective amount. A prophylactically effective amount of a B. formosana extract means an amount of a therapeutic agent, alone or in combination with other agents, which provides a prophylactic benefit in the prevention of the condition. For example, a prophylactically effective amount of B. formosana extract can be an amount sufficient to prevent or delay the onset of a condition, or one or more symptoms associated with the condition or prevent its recurrence. It may also be an amount that improves overall prophylaxis or enhances the prophylactic efficacy of another prophylactic agent. Furthermore, the effective amount can be a human equivalent dose (HED) converted from an animal dose used in the working examples of the present disclosure in accordance with Industry Guidance issued by the Food and Drug Administration (Guidance for Industry: Estimating the Maximum Safe Starting Dose in Initial Clinical Trials for Therapeutics in Adult Healthy Volunteers, U.S. Department of Health and Human Services, Food and Drug Administration Center for Drug Evaluation and Research (CDER), July 2005).

A “subject” as described herein can be a human subject (e.g., a pediatric subject such as an infant, a child, or an adolescent, or an adult subject such as a young adult, middle-aged adult, or senior adult), or a non-human animal, such as dogs, cats, cows, pigs, horses, sheep, goats, rodents (e.g., mice, rats), and non-human primates (e.g., cynomolgus monkeys, rhesus monkeys). The non-human mammal may be a transgenic animal or genetically engineered animal. In some embodiments, the subject is a human patient having a target disease as described herein (e.g., ARDS, diabetes mellitus, psoriasis, liver injury, and etc.), suspected of having the disease, or is at risk for the disease. In other embodiments, the subject is a human or non-human mammal having, suspected of having a condition secondary to dysregulated activation of neutrophils (e.g., ARDS, diabetes mellitus, psoriasis, acute liver injury, and etc.).

Notwithstanding that the numerical ranges and parameters setting forth the broad scope of the invention are approximations, the numerical values outlined in the specific examples are reported as precisely as possible. Any numerical value, however, inherently contains certain errors necessarily resulting from the standard deviation found in the respective testing measurements Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values, and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Use of the B. formosana Extract of the Present Invention

The present disclosure lies in the unexpected discovery of a Bletilla formosana extract prepared in accordance with the process described herein that possesses therapeutic effects toward dysregulated activated human neutrophils. Accordingly, the Bletilla formosana extract may be used as a candidate agent for the development of medicaments suitable for treating diseases or disorders associated with dysregulated activation and recruitment of neutrophils, such as ARDS, ALI, diabetes mellitus, psoriasis, and the like.

Accordingly, it is the first aspect of the present disclosure to provide a method of treating a subject suffering from ARDS, ALI, diabetes mellitus, or psoriasis. The method comprises administering to the subject an effective amount of a Bletilla formosana extract.

Preferably, the Bletilla formosana extract of the present invention is prepared by a method comprising steps of, (i) extracting Bletilla formosana bulbs, leaves, stems or a combination thereof with water to produce a first extract and a first residue; and (ii) extracting the first residue of step (i) with ethyl acetate to produce the Bletilla formosana extract; and the thus produced Bletilla formosana extract is termed “−W+EA” extract in the present disclosure. Alternatively, the Bletilla formosana extract of the present invention may be prepared by extracting Bletilla formosana bulbs, leaves, stems or a combination thereof with ethyl acetate, and the thus produced Bletilla formosana extract is termed “EA” extract in the present disclosure.

Bioactivity analysis of the present Bletilla formosana extract indicates that it is a powerful inhibitory agent toward superoxide anion production, elastase release, reactive oxygen species (ROS) production, and degranulation in stimulated human neutrophils. Furthermore, the Bletilla formosana extract does not affect cell viability. HPLC/MS analysis of the Bletilla formosana extract indicates that it includes, at least, compounds of 3,3′-dihydroxy-5-methoxybibenzyl (Batatacin III), 9,10-dihydro-1-[(4-hydroxyphenyl)methyl]-4-methoxy-2,7-phenanthrenediol (Orchidble), 3′,5-dimethoxy-3-hydroxybibenzyl (BF8-4-2), 3,5-dimethoxy-3′-hydroxybibenzyl (BF8-4-3) and 3-hydroxy-5-methoxybibenzyl (BF8-4-4). Bioactivity analysis on the compounds identified in the present Bletilla formosana extract confirms that every one of them is capable of suppressing superoxide anion production from activated neutrophils, and three of them, except BF8-4-2, could suppress elastase release from activated neutrophils. Findings of the present disclosure confirm that the present Bletilla formosana extract (e.g., the “EA” or “−W+EA” extracts) may serve as a candidate agent for the development of medicaments suitable for treating diseases associated with dysregulated activation and recruitment of neutrophils, such as ARDS, ALI, diabetes mellitus, psoriasis, and the like.

According to embodiments of the present disclosure, ARDS may be resulted from transfusion-related lung injury, ventilator-induced lung injury, bacteria-induced lung injury, viral-induced lung injury, etc.

According to embodiments of the present disclosure, the Bletilla formosana extract (e.g., “EA” or “−W+EA” extracts), is administered to the subject in the amount of 0.01 to 1,000 mg/kg, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1,000 mg/kg; preferably, the Bletilla formosana extract is administered to the subject in the amount of 0.1 to 800 mg/kg, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, and 800 mg/kg; more preferably, the Bletilla formosana extract is administered to the subject in the amount of 1 to 100 mg/kg, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 mg/kg. In one preferred embodiment, the Bletilla formosana extract is administered to the subject in the amount of 4 mg/kg. In another preferred embodiment, the Bletilla formosana extract is administered to the subject in the amount of 8 mg/kg. The effective amount of a compound may be administered in one or more doses for one or several days (depending on the mode of administration).

The present Bletilla formosana extract may also be formulated with suitable carriers or excipients for a suitable administration route, e.g., orally, parenterally, by inhalation spray, topically, rectally, nasally, buccally, vaginally or via an implanted reservoir. The term “parenteral” includes subcutaneous, intracutaneous, intravenous, intramuscular, intraarticular, intraarterial, intrasynovial, intrasternal, intrathecal, intralesional and intracranial injection or infusion techniques.

A sterile injectable formulation, e.g., a sterile injectable aqueous or oleaginous suspension, can be formulated according to techniques known in the art using suitable dispersing or wetting agents (such as TWEEN® 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution, and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A formulation suitable for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the Bletilla formosana extract of the present disclosure can be suspended or dissolved in an oily phase combined with emulsifying or suspending agents. If desired, certain sweetening, flavoring, or coloring agents can be added. A nasal aerosol or inhalation formulation can be prepared according to techniques well-known in the art of pharmaceutical formulation and can be prepared as solutions in saline, employing benzyl alcohol or other suitable preservatives, absorption promoters to enhance the bioavailability, fluorocarbons, and/or other solubilizing or dispersing agents known in the art. The Bletilla formosana extract of the present disclosure can also be administered in the form of suppositories for rectal administration.

Pharmaceutically acceptable carriers or excipients that may be included in a formulation comprising the Bletilla formosana extract of the present disclosure include inert diluents, solubilizing agents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

An excipient present in an inventive formulation must be “pharmaceutically acceptable” in the sense that the excipient is compatible with the active ingredient of the pharmaceutical composition (and preferably, capable of stabilizing the pharmaceutical composition) and not deleterious to a subject to whom the pharmaceutical composition is administered. For example, solubilizing agents such as cyclodextrins, which may form specific, more soluble complexes with the Bletilla formosana extract of the invention, can be utilized as pharmaceutically acceptable excipients for delivery of the Bletilla formosana extract of the invention into the subject. Examples of other pharmaceutically acceptable excipients include colloidal silicon dioxide, magnesium stearate, cellulose, sodium lauryl sulfate, etc.

Also disclosed herein are kits (e.g., pharmaceutical packs) comprising the Bletilla formosana extract described herein, and a container (e.g., a vial, ampule, bottle, syringe, and/or dispenser package, or other suitable containers). In some embodiments, the kits may include a second container comprising a pharmaceutically acceptable excipient for dilution or suspension of an inventive formulation. In some embodiments, the Bletilla formosana extract provided in the first container and the pharmaceutically acceptable excipient (e.g., saline) provided in the second container are combined to form one-unit dosage form.

In certain embodiments, a kit as described herein is for use in inhibiting the dysregulated activation and recruitment of neutrophils. In certain embodiments, a kit as described herein is for use in treating any of the target diseases as described herein (e.g., ARDS, ALI, diabetes mellitus, psoriasis) in a subject in need thereof. Any of the kits described herein can thus include instructions for administering the Bletilla formosana extract contained therein. A kit of the invention may also include information as required by a regulatory agency such as the FDA. In certain embodiments, the kit and instructions provide for treating a disease described herein. A kit of the invention may include one or more additional pharmaceutical agents described herein as a separate composition.

It will be also appreciated that an extract or formulation, as described herein, can be used in combination with one or more additional agents (e.g., therapeutically and/or prophylactically active agents) in any of the methods described herein. The Bletilla formosana extract or formulation can be administered in combination with additional agents that improve their activity (e.g., activity (e.g., potency and/or efficacy) in treating a disease described herein in a subject in need thereof, in preventing a disease described herein in a subject in need thereof, in inhibiting the activation of neutrophile in a subject. It will also be appreciated that the therapy employed may achieve a desired effect for the same disorder, and/or it may achieve different effects.

The present invention will now be described more specifically with reference to the following embodiments, which are provided for the purpose of demonstration rather than limitation. While they are typical of those that might be used, other procedures, methodologies, or techniques known to those skilled in the art may alternatively be used.

### EXAMPLES

### Materials and Methods

Preparation of Bletilla formosana Extract

Bletilla formosana bulbs, leaves, stems, or a mixture thereof were dried and grounded before being stored at −20° C. until use. The grounded powders of Bletilla formosana (10 g) were extracted by any one of the solvents, including n-hexane, n-hexane/ethyl acetate (1:1), n-hexane/ethyl acetate (3:1), ethyl acetate, 95% ethanol, and double-distilled water (ddH₂O) at room temperature to give a corresponding crude extract. A total of 6 crude extracts were produced and were independently termed “H,” “1H1E,” “3H1E,” “EA”, “EtOH,” and “H₂O” extracts. The residue of the H₂O extract was further extracted with ethyl acetate to give the extract that was termed “−W+EA” extract.

Each of the six crude extracts or the “−W+EA extract” was sonicated (30 min), filtered, and condensed to give a corresponding Bletilla formosana (B. formosana ) extract. Each B. formosana extract was subjected to liquid chromatography/mass spectrum analysis (LC-QTOF-MS/MS) to identify active components therein.

### Human Neutrophil Isolation

The study was conducted with the approval of the Institutional Review Board of Chang Gung Memorial Hospital in accordance with the Declaration of Helsinki. After written informed consent was obtained, whole blood samples were drawn from healthy individuals aged 20-30 years who had not taken any medication within the previous 5 weeks. Neutrophils were then isolated using the standard procedures for dextran sedimentation, Ficoll-Hypaque gradient centrifugation, and hypotonic lysis of erythrocytes. The isolated neutrophils were then suspended in Ca²⁺-free HBSS (pH 7.4) and stored at 4° C. until use.

### Analysis of Neutrophil Elastase (NE) Release

Human neutrophils (6×10⁵ cells/mL) were incubated with DMSO or the present B. formosana extract after treatment with 1 mM CaCl₂ and 100 μM NE substrate (Methoxysuccinyl-Ala-Ala-Pro-Val-p-nitroanilide) at 37° C. for 5 min. Cells were stimulated with fMLF (0.1 μM)/cytochalasin B (CB) (0.5 μg/mL) for 10 min before determination of NE release by measuring the change of absorbance at 405 nm in a spectrophotometer.

### Analysis of Neutrophil Extracellular Trap (NET) Formation

Neutrophils (2×10⁶ cells/mL) were incubated with DMSO or the present B. formosana extract for 5 min before being activated with 10 nM PMA for 3 h, followed by the addition of deoxyribonuclease (DNase) (2 U/mL) for 10 min. The reaction was terminated by the addition of EDTA (2 mM) at 4° C., the thus produced mixture was then centrifuged at 4° C. for 5 min. The supernatant was collected and mixed with SYTOX Green (5 μM) in a 96-wells plate. fluorescence image system was used to evaluate the NET formation of activated neutrophils by the emitted fluorescence in each 96-wells.

### Measurement of Extracellular Superoxide Anion Production

Extracellular superoxide anion production in activated neutrophils was assessed through the reduction of ferricytochrome c. After incubating them with Ca²⁺(1 mM) and ferricytochrome c (0.6 mg/mL) at 37° C., the isolated human neutrophils (6×10⁵ cells/mL) were then incubated with DMSO or the present B. formosana extract for 5 min. The cells were pretreated with cytochalasin B (CB, 1 or 2 μg/mL) for 3 min and stimulated with fMLF. The change in absorbance at 550 nm was detected continuously using a spectrophotometer (U-3010, Hitachi, Tokyo, Japan), and superoxide anion levels were calculated using a method described previously (Hwang et al., 2003 Mol. Pharmacol. 64(6), 1419-1427).

### Analysis of Total ROS Production

Human neutrophils (2×10⁶ cells/mL) were preincubated with 6 U/mL horseradish peroxidase (HRP) and 37.5 μM luminol in a 96-well plate at 37° C. for 5 min. Cells were incubated with DMSO or B. formosana extract for 5 min, followed by 0.1 μM fMLF stimulation. Chemiluminescence was then detected and analyzed in real-time on a 96-well chemiluminometer (Tecan Infinite F200 Pro; Männedorf, Switzerland).

### Cell Viability Test

Cytotoxicity of B. formosana extract towards neutrophils was evaluated by measuring the level of lactate dehydrogenase (LDH) using a commercial kit (Promega). LDH is a cytosolic enzyme that is released only when cell membrane is disrupted (i.e., cell death), thus may be used as an indicator of cell viability. The kit measures a color change at (λmax=450 nm) when LDH reduces NAD to NADH. Cytotoxicity was expressed as the percent LDH activity obtained in a cell-free medium compared to the total LDH activity. Total LDH activity was determined by lysing cells with 0.1% Triton X-100 for 30 min at 37° C.

### Animals

The animal care and experiment protocols were approved by the Institutional Animal Care and Use Committee of Chang Gung University, Taiwan. Moreover, the animal studies were reported in accordance with the ARRIVE (Animal Research:Reporting of In Vivo Experiments) guidelines. All the experimental procedures complied with The Guide for the Care and Use of Laboratory Animals (National Research Council Committee for the Update of the Guide for the Care and Use of Laboratory, 2011). Specified pathogen-free (SPF) 8-week-old male BALB/c mice (body weight: 20±1 g) were purchased from BioLASCO (Taiwan). Five mice shared a ventilated cage with standard bedding, and were given ad libitum access to water and standard laboratory chow. All mice were kept in an SPF animal facility under a 12-12-h light-dark cycle. Mice were acclimatized for at least 1 week before use in the experiments.

### Imiquimod (IMQ)-Induced Psoriasis in Mice

BALB/c mice were pre-treated with IMQ (62.5 mg) for 60 min by topically administered onto the back of the mice every day for 5 days (i.e., days 0 to 4). The present B. formosana extract (50 mg/kg) was also topically administered onto the IMQ-treated skin area for 60 min prior to the daily IMQ treatment on day 2 for 4 days (i.e., days 1 to 4). Mice were sacrificed on day 5.

### LPS-Induced Acute Respiratory Distress Syndrome (ARDS) in Mice

Male BALB/c mice were randomly divided into four groups (2-3 mice/group): vehicle alone, B. formosana extract alone, LPS control, and B. formosana extract treatment (B. formosana extract+LPS). The mice were starved overnight and then intraperitoneally injected with 50 μL of B. formosana extract (50 mg/kg) or 50 μL of vehicle (10% DMSO). ARDS was induced through intratracheal spraying of 50 μL of LPS (from Escherichia coli O111:B4; 2 mg/kg) or 50 μL of 0.9% saline (in vehicle and B. formosana extract alone group) under general anesthesia with xylazine (6 mg/kg) and Zoletil 50 (30 mg/kg). Five hours later, mice were sacrificed, thereafter the lungs were harvested and fixed with 10% paraformaldehyde for histological sectioning.

### Histological Sectioning and Staining

The harvested lung tissues were washed with phosphate-buffered saline (PBS) and fixed with 10% formalin for 24 h. The samples were subsequently dehydrated, embedded with paraffin, sliced into 3-μm-thick sections with a microtome, and placed on glass slides. These sections were stained using haematoxylin and eosin (H&E). Then, images were acquired through light microscopy.

### Combined Treatment of D-GalN and LPS Induced Acute Liver Injury (ALI) in Mice

Male BALB/c mice were randomly divided into four groups (2-3 mice/group): vehicle alone, LPS+D-GalN (LPS, 40 μg/mL; D-galactosamine, 400 mg/kg) control, LPS+D-GalN+“−W+EA” extract (50 mg/Kg) group A, and LPS+D-GalN+“−W+EA” extract (100 mg/kg) group B. Each mouse was intraperitoneally injected with 10% DMSO or LPS+D-GalN for 1 hr, followed by intraperitoneal injection of the “−W+EA” extract for another 5 hr. Blood samples were drawn and analyzed for the level of glutamate pyruvate transaminase (GPT) and glutamate oxaloacetic transaminase (GOT) therein. Mice were then sacrified and their liver tissues were harvested and analyzed by H&E staining.

### Streptozotocin (STZ)-Induced Diabetic Mice and Treatment

Wide-type male C57BL/6 mice were rendered diabetic by intraperitoneal injection of STZ (50 mg/kg/day) for 5 days, from day 1 to day 5. Mice exhibited an averaged blood glucose level greater than 230 mg/dL on day 6 were selected for subsequent study. The vehicle or “−W+EA” extract (25 mg/Kg) was intraperitoneally injected into each animal daily from day 9, and continued for 5 days, from day 9 to day 13. Fasting blood glucose (i.e., blood glucose level after fasting for 8 hrs) and body weight of each mouse were measured from day 9 to day 13.

### Example 1 In Vitro Characterization of the Present B. formosana Extract

### 1.1 B. formosana Extract Suppressed Superoxide Anion Production and Elastase Release in Stimulated Neutrophils

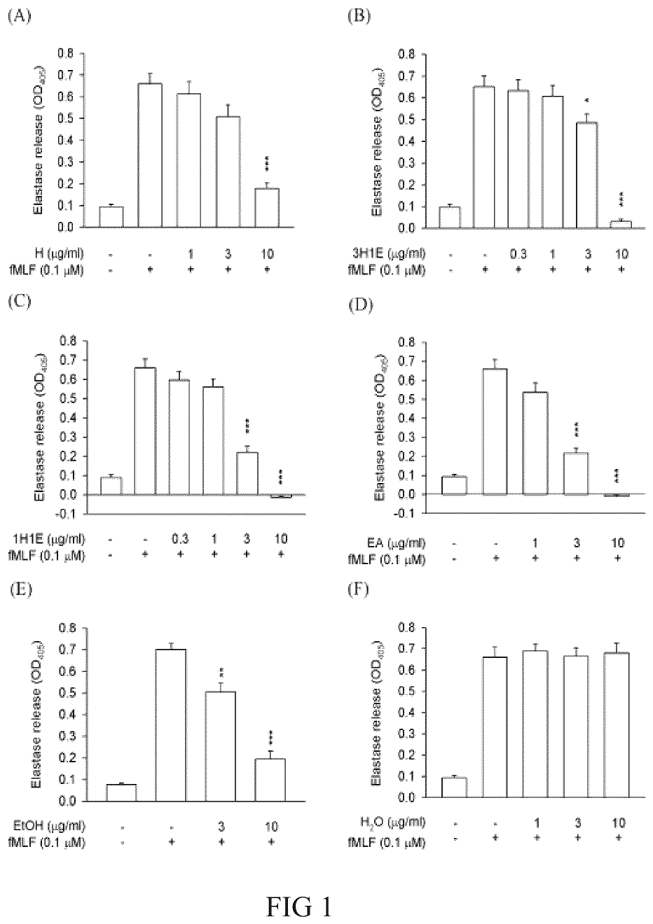

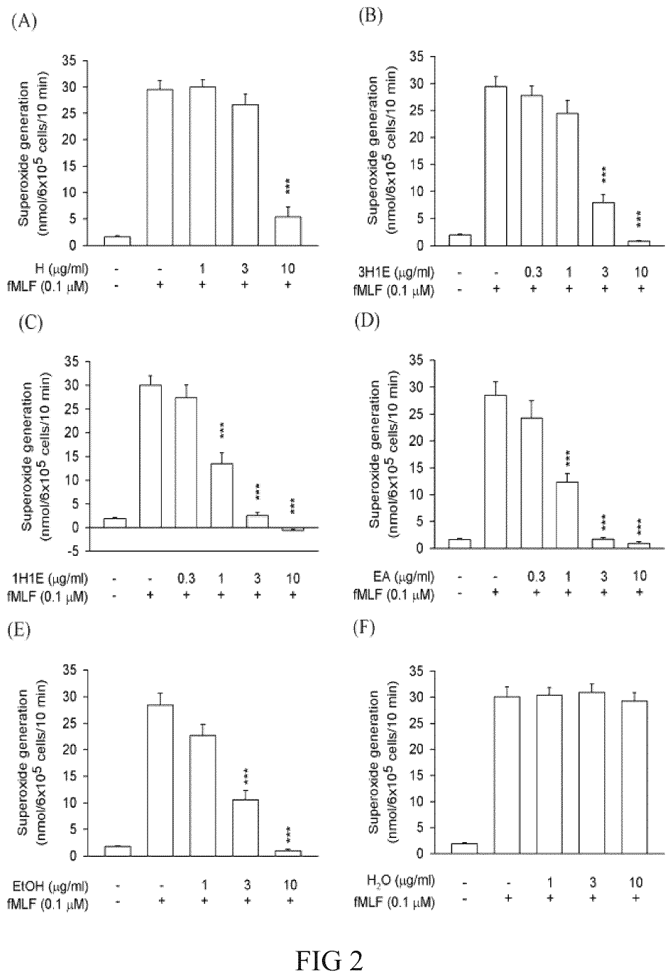

B. formosana extracts were prepared in accordance with procedures described in the “Materials and Methods” section. A total of seven B. formosana extracts were produced, and were independently tested for its effect on inflammatory responses via monitoring the production of superoxide anions and elastase release in human neutrophils stimulated with formyl-L-methionyl-L-leucyl-L-phenylalanine (fMLF)/cytochalasin B (CB). Results are provided in Table 1, and FIGS. 1 and 2.

TABLE 1
Effects of B. formosana extracts on superoxide anions
elastase release in fMLF/CB-activated human neutrophils

| | Superoxide anions | | | Elastase release | | | |
|---|---|---|---|---|---|---|---|
| Name of | IC₅₀ | Inhibition | | IC₅₀ | Inhibition | | Extraction |
| extract | (μg/mL)^{a} | (%)^{b} | | (μg/mL)^{a} | (%)^{b} | | (%) |
| H | 6.06 ± 0.49 | 76.91 ± 7.44 | *** | 4.74 ± 0.35 | 85.12 ± 3.28 | *** | 0.68 |
| 3H1E | 1.94 ± 0.16 | 97.09 ± 0.70 | *** | 3.98 ± 0.15 | 113.47 ± 2.81 | *** | 1.90 |
| 1H1E | 0.98 ± 0.13 | 102.02 ± 0.65 | *** | 1.86 ± 0.14 | 120.23 ± 3.93 | *** | 2.90 |
| EA | 0.79 ± 0.08 | 96.89 ± 1.26 | *** | 1.71 ± 0.11 | 120.54 ± 4.98 | *** | 3.49 |
| EtOH | 2.19 ± 0.19 | 96.62 ± 0.84 | *** | 4.80 ± 0.55 | 82.77 ± 5.65 | *** | 12.29 |
| H₂O | >10 | 1.63 ± 4.29 | | >10 | −4.30 ± 2.09 | | 31.81 |

^{a}Concentration necessary for 50% inhibition.
^{b}Percentage of inhibition at 10 μg/mL.
Results are presented as mean ± S.E.M. (n = 6~7).
* p < 0.05, ** p < 0.01, *** p < 0.001 compared with that of the control (DMSO).

According to Table 1, solvents with higher polarity exhibited higher extraction power for B. formosana , in which water exhibited an extraction rate of 31.81% (“H₂O extract”), which was followed by 95% ethanol with an extraction rate of 12.29% (“EtOH extract”). By contrast, non-polar solvent such as n-hexane had minimum extraction power with an extraction rate as low as 0.68% (“H extract”).

As to the modulatory effect of each extract, it was found that except the water extract (“H₂O extract”), the other extracts including extracts of n-hexane/ethyl acetate (1:1) (“1H1E extract”), n-hexane/ethyl acetate (3:1) (“3H1E extract”), ethyl acetate (“EA extract”), and 95% ethanol (“EtOH extract”), all could suppress the production of superoxide anions and elastase release in fMLF/CB-activated human neutrophils (FIGS. 1 and 2). Among these extracts, the “EA extract” exhibited the strongest inhibitory effect with an IC₅₀ of 0.79 μg/mL on superoxide anions production, and an IC₅₀ of 1.71 μg/mL on elastase release (Table 1).

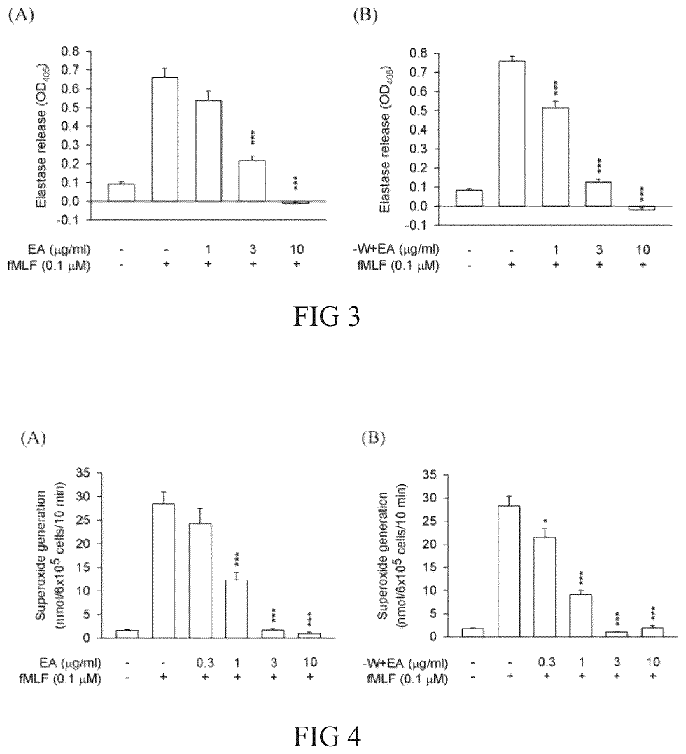

As water exhibited the highest extraction power and most anti-inflammatory components of B. formosana were found in the “EA extract,” thus, the residue of the “H₂O extract” was further extracted with EA to produce the “−W+EA extract.” The modulatory effects of “EA extract” and “−W+EA extract” on fMLF/CB-activated human neutrophils are provided in Table 2 and FIGS. 3 and 4.

TABLE 2
Effects of different B. formosana extracts on superoxide anions
elastase release in fMLF/CB-activated human neutrophils

| | Superoxide anions | | | Elastase release | | | |
|---|---|---|---|---|---|---|---|
| Name of | IC₅₀ | Inhibition | | IC₅₀ | Inhibition | | Extraction |
| extract | (μg/mL)^{a} | (%)^{b} | | (μg/mL)^{a} | (%)^{b} | | (%) |
| EA | 0.79 ± 0.08 | 96.89 ± 1.26 | *** | 1.71 ± 0.11 | 120.54 ± 4.98 | *** | 3.49 |
| W + EA | 0.61 ± 0.04 | 92.93 ± 1.73 | *** | 2.19 ± 0.19 | 96.62 ± 0.84 | *** | 2.89 |

^{a}Concentration necessary for 50% inhibition.
^{b}Percentage of inhibition at 10 μg/mL.
Results are presented as mean ± S.E.M. (n = 6~7).
* p < 0.05, ** p < 0.01, *** p < 0.001 compared with that of the control (DMSO).

It was found that “−W+EA extract” exhibited similar modulatory effects as those of the “EA extract” in terms of inhibition of superoxide anions production and elastase release (Table 2, and FIGS. 3 and 4).

### 1.2 B. formosana Extracts does not Affect Cell Viability

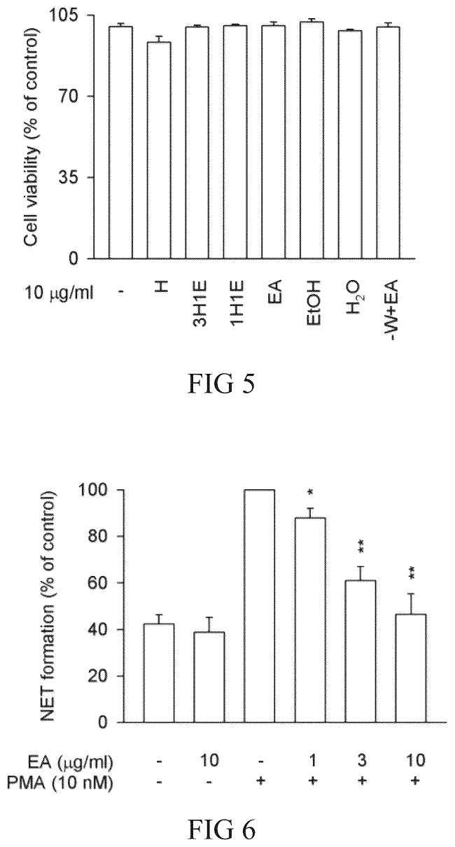

In this example, whether the B. formosana extracts of Example 1.1 affect cell viability was investigated via monitoring the level of lactate dehydrogenase (LDH) released from the cells. LDH is a cytosolic enzyme that is released only when cell membrane is disrupted (i.e., cell death), thus may be used as an indicator of cell viability. Results are depicted in FIG. 5.

It was evident that none of the seven B. formosana extracts of Example 1.1 had any cytotoxicity, as the level of LDH released from the B. formosana extract treated human neutrophils was negligible.

### 1.3 B. formosana EA Extract Attenuated Neutrophil Extracellular Trap (NET) Formation

NET, mainly composed of granular proteins, proteases, and chromatin filaments coated with histones, is crucial in inflammatory diseases and autoimmune disorders. To investigate the effects of B. formosana EA extract on NET formation, neutrophils were stained with Sytox green after activation with PMA (10 nM).

As depicted in FIG. 6, PMS-induced NET formation was significantly mitigated by B. formosana EA extract.

### 1.4 B. formosana EA Extract Ameliorated ROS Production in Stimulated Neutrophils

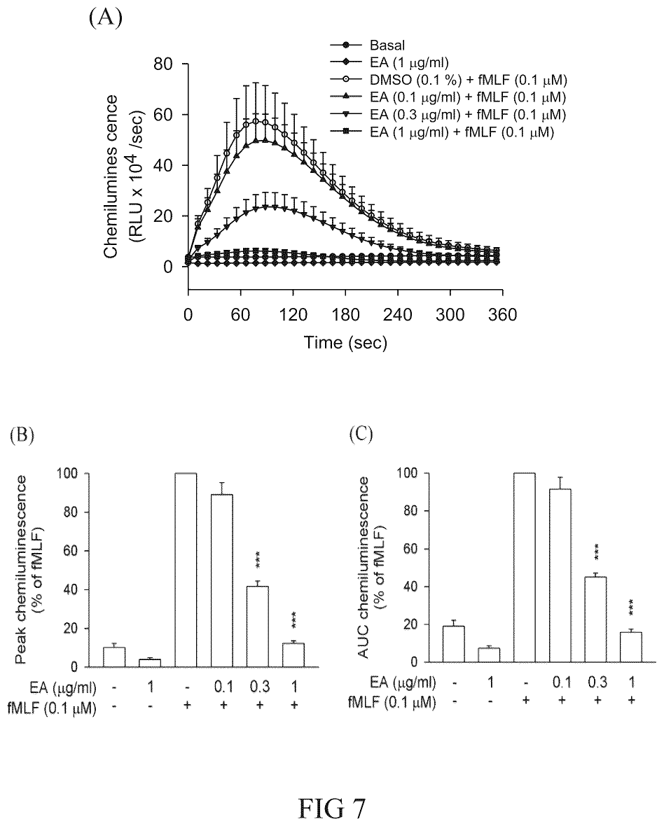

In this example, whether the B. formosana EA extract affected ROS production in stimulated neutrophils was investigated via flow cytometry and chemiluminescence assay. Results are illustrated in FIG. 7.

The quantified results of flow cytometry and luminol-amplified chemiluminescence assay revealed that B. formosana EA extract significantly suppressed intracellular ROS production in fMLF-activated neutrophils in a dose-dependent manner (FIG. 7).

### 1.5 Chemical Components of B. formosana Extract

Each B. formosana extract of Example 1.1 was subjected to LC/MS analysis to identify chemical components therein, and the modulatory effects of each identified components on superoxide anions production and elastase release in activated neutrophils were further investigated. Results are summarized in Tables 3 and 4.

Chemical components including Batatasin III, Orchidble, BF8-4-2, BF8-4-3, and BF8-4-4 were present in the “3H1H,” “1H1E,” “EA,” and “−W+EA” extracts (Table 3), and each component was found to suppress the superoxide anions production and elastase release in activated human neutrophils (Table 4).

TABLE 3
Contents of chemical components of
B. formosana in various extracts

| Compound | H | 3H1E | 1H1E | EA | EtOH | H₂O | −W + EA |
|---|---|---|---|---|---|---|---|
| Batatasin III | 0.06 | 3.67 | 2.72 | 0.93 | 0.93 | 0.10 | 1.39 |
| Orchidble | N.D.^{a} | 1.12 | 1.25 | 0.41 | 0.41 | 0.04 | 1.77 |
| BF8-4-2 | 1.91 | 1.10 | 0.72 | 0.64 | 0.12 | N.D.^{a} | 0.39 |
| BF8-4-3 | 1.78 | 0.92 | 0.61 | 0.53 | 0.12 | N.D.^{a} | 0.38 |
| BF8-4-4 | 2.8 | 1.10 | 0.67 | 0.60 | 0.14 | N.D.^{a} | 0.37 |

^{a}Not detected.

TABLE 4
Effects of chemical components of B. formosana extract on superoxide
anions elastase release in fMLF/CB-activated human neutrophils

| | Superoxide anions | Elastase release |
|---|---|---|
| Compound | IC₅₀ (μM) | IC₅₀ (μM) |
| Batatasin III | 4.30 ± 0.50 | >10 |
| Orchidble | 0.66 ± 0.12 | 6.26 ± 1.19 |
| BF8-4-2 | 4.63 ± 0.54 | >10 |
| BF8-4-3 | 4.64 ± 0.23 | 7.41 ± 0.50 |
| BF8-4-4 | 1.86 ± 0.21 | 2.71 ± 0.16 |

### Example 2 B. formosana Extract Alleviated Imiquimod (IMQ)-Induced Psoriasis in Mice

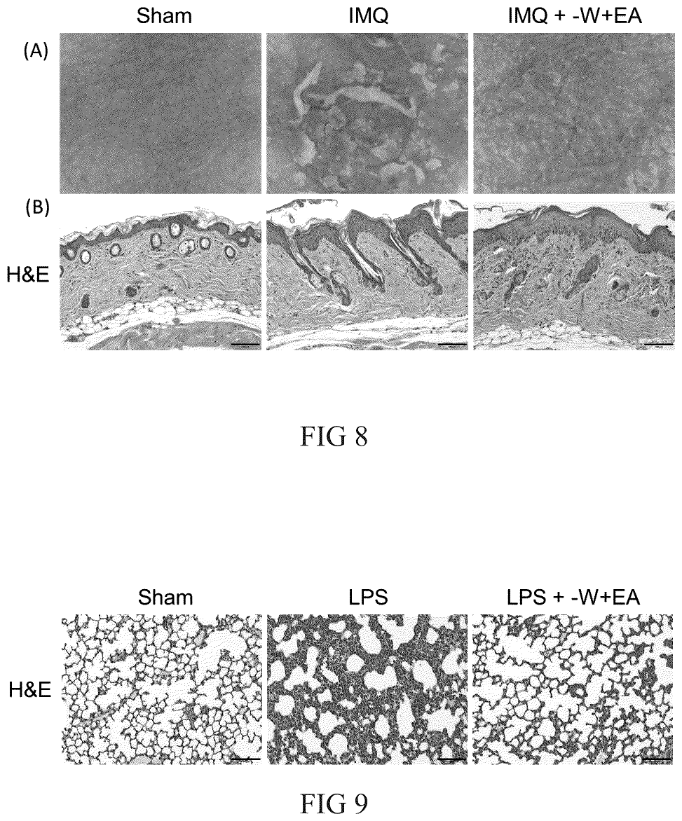

In this example, the effect of B. formosana extract on the development of psoriasis was investigated using the well-established psoriasis animal model, in which imiquimod (IMQ) was used to induce psoriasis-like skin inflammation. Results are depicted in FIG. 8.

FIG. 8(A) are photographs taken from one representative mouse treated with IMQ (center), and IMQ+“−W+EA” extract (right). FIG. 8(B) are microscopic presentations of skins taken from the control IMQ-treated mouse, and the mouse treated with IMQ+“−W+EA” extract. It is evident that the present B. formosana “−W+EA” extract significantly reduces the severity or progression of IMQ-induced psoriasis in the tested animals.

### Example 3 B. formosana Extract Alleviated Lipopolysaccharide (LPS)-Induced Acute Respiratory Distress Syndrome in Mice

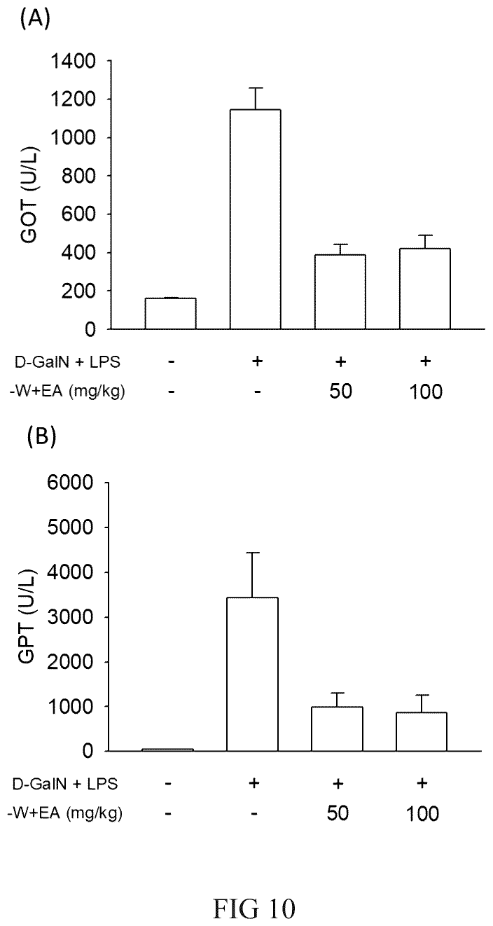

In this example, the effect of B. formosana extract on LPS-induced acute respiratory distress syndrome was investigated. To this purpose, BALB/c mice were treated with the “−W+EA” extract (50 mg/kg) or DMSO administered through intraperitoneal injection followed by intratracheal spraying of LPS for 5 h. The exterior photos and HE-stained histopathological features of lungs revealed that LPS induced haemorrhagic and erythematous conditions, interalveolar septal thickening, and pulmonary interstitial oedema formation (FIG. 9). Distortion of the pulmonary architecture was significantly suppressed in the “−W+EA” extract treatment group.

### Example 4 B. formosana Extract Ameliorated D-GalN/LPS-Induced Acute Liver Injury in Mice

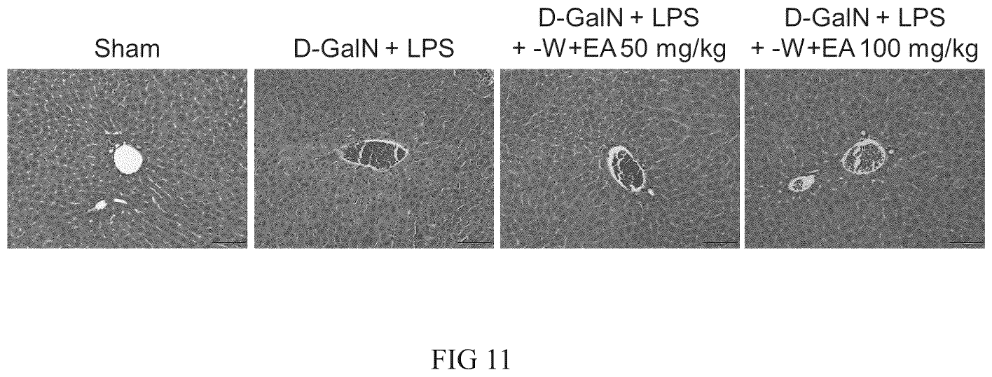

In this example, the effect of B. formosana extract on D-GalN/LPS-induced acute liver injury was investigated. To this purpose, BALB/c mice were intraperitoneally injected with D-GalN (400 mg/Kg) and LPS (40 μg/mL) or saline (220 μL) for 1 hr, then with the “−W+EA” extract (50 or 100 mg/kg) for another 5 hrs. Blood samples were drawn and plasma were analyzed for the levels of GOT and GPT enzymes; while liver tissues were collected and subjected to H&E staining. Results are provided in FIGS. 10 and 11.

FIG. 10 are bar graphs depicting the effect of “−W+EA” extract on the levels of GOT and GPT, and FIG. 11 are photographs of the liver tissues in the control, D-GalN/LPS-treated and D-GalN/LPS+”−W+EA” extract treated mice.

It was clear that D-GalN/LPS caused significant damage to liver tissue (FIG. 11) with elevated levels of GOT and GPT found in the sera, and these elevated levels of GOT and GPT were effectively reduced by the treatment of the “−W+EA” extract (FIG. 10).

### Example 5 B. formosana Extract Reduced STZ-Induced Blood Glucose Elevation in Mice

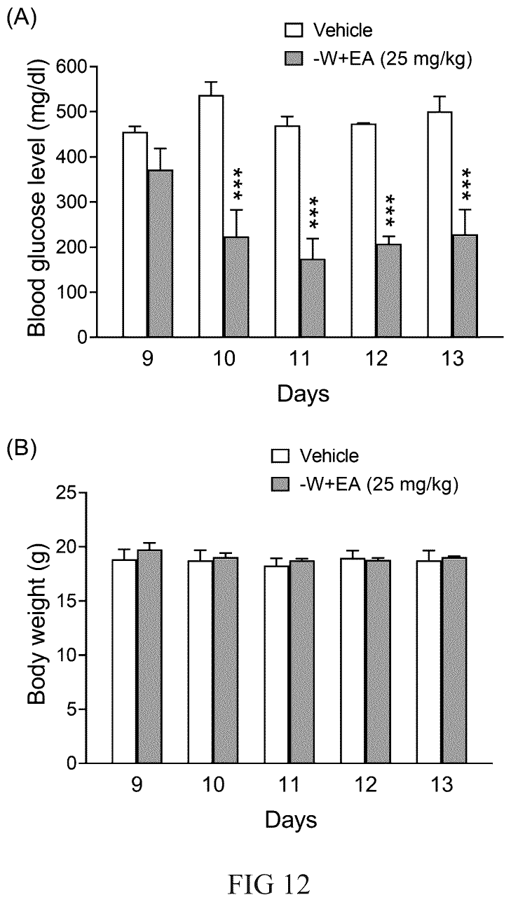

In this example, the effect of B. formosana extract on STZ-induced blood glucose elevation was investigated. For this purpose, C57B6 mice were treated with STZ (50 mg/Kg) to induce elevated blood glucose level in accordance with procedures described in the “Materials and Methods” section. The diabetic-like animals were then treated with the “−W+EA” extract (25 mg/kg) or saline for another 5 days. Results are provided in FIG. 12.

From the data in FIG. 12, it was clear that the “−W+EA” extract at the daily dose of 25 mg/kg could effectively reduce STZ-introduced elevation in blood glucose level without adversely affecting the body weight of the test animals.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the scope of the present disclosure.

1. A method of treating a subject having acute respiratory distress syndrome (ARDS), acute liver injury (ALI), diabetes mellitus or psoriasis comprising administering to the subject an effective amount of an extract of Bletilla formosana, which comprises 3,3′-dihydroxy-5-methoxybibenzyl (Batatacin III), 9,10-dihydro-1-[(4-hydroxyphenyl)methyl]-4-methoxy-2,7-phenanthrenediol (Orchidble), 3′,5-dimethoxy-3-hydroxybibenzyl (BF8-4-2), 3,5-dimethoxy-3′-hydroxybibenzyl (BF8-4-3) and 3-hydroxy-5-methoxybibenzyl (BF8-4-4).

2. The method of claim 1, wherein the Bletilla formosana extract is prepared by,
(i) extracting Bletilla formosana bulbs, leaves, stems or a mixture thereof with water to produce a first extract and a first residue; and
(ii) extracting the first residue of step (i) with ethyl acetate to produce the Bletilla formosana extract.

3. The method of claim 1, wherein the Bletilla formosana extract is prepared by extracting Bletilla formosana bulbs, leaves, stems or a mixture thereof with ethyl acetate.

4. The method of claim 1, wherein the Bletilla formosana extract is administered to the subject in the amount of 0.01 to 1,000 mg/Kg/day.

5. The method of claim 4, wherein the ARDS is transfusion-related lung injury, ventilator-induced lung injury, bacteria-induced lung injury, or virus-induced lung injury.

6. The method of claim 1, wherein the subject is a mammal.

7. The method of claim 6, wherein the subject is a human.
